(19) United States
(12) Patent Application Publication
(10) Pub. No.: US 2015/0201899 A1
(43) Pub. Date: Jul. 23, 2015

(54) IMAGING SUPPORT SYSTEM AND MEDICAL IMAGE IMAGING APPARATUS

(71) Applicants: Hitoshi Uchinomiya, Tokyo (JP); Tadashi Nakamura, Tokyo (JP); Konosuke Temmei, Tokyo (JP); Masakazu Okabe, Tokyo (JP); Yuji Sakai, Tokyo (JP)

(72) Inventors: Hitoshi Uchinomiya, Tokyo (JP); Tadashi Nakamura, Tokyo (JP); Konosuke Temmei, Tokyo (JP); Masakazu Okabe, Tokyo (JP); Yuji Sakai, Tokyo (JP)

(73) Assignee: Hitachi Medical Corporation, Tokyo (JP)

(21) Appl. No.: 14/419,160

(22) PCT Filed: Sep. 11, 2013
(86) PCT No.: PCT/JP2013/074486

§ 371 Date: Feb. 2, 2015

(30) Foreign Application Priority Data
Sep. 19, 2012 (JP) 2012-205730

(51) Int. Cl. A61B 6/00; A61B 6/04

(52) U.S. Cl. CPC A61B 6/547; A61B 6/0407; A61B 6/563

(57) ABSTRACT

In order to provide a technique for simplifying pre-examination input tasks for a medical image imaging apparatus, an imaging support system includes the medical image imaging apparatus 1 generating a medical image of the object 4, the position calculation unit 111 detecting a current position of the medical image imaging apparatus 1, the object candidate search unit 116 searching object candidates to be examined based on the current position, the operation unit 31 receiving a selection operation of an object to be examined from among object candidates, and the imaging condition setting unit 93 setting imaging conditions of the object to be examined.

### TECHNICAL FIELD

The present invention relates to an imaging support system and a medical image imaging apparatus, in particular, to a technique for inputting parameters such as imaging conditions.

### BACKGROUND ART

In the patent literature 1, as an example of an X-ray diagnostic apparatus that automatized settings such as an operation parameter, an X-ray diagnostic apparatus providing a reference level range of an expected pixel value output from an X-ray flat surface detector, comparing a pixel value of an X-ray image in case of irradiating an X ray with the reference level range of the expected pixel value, determining an operation parameter automatically within the reference level range of the expected value based on the comparison result, and automatically determining an offset correction coefficient to correct an offset component of the X-ray flat surface detector 12; a gain correction coefficient; and position information of defect points is disclosed.

### CITATION LIST

### Patent Literature

PTL 1: Japanese Unexamined Patent Application Publication No. 2003-244540

### SUMMARY OF INVENTION

### Technical Problem

According to the above X-ray diagnostic apparatus, although operation parameters such as an offset correction coefficient can be input automatically, there was a problem in which X-ray conditions, for example, a parameter such as mA, kV, and mAs that were required to irradiate an X-ray of an amount corresponding to each procedure and/or each object still had to be manually input by an operator.

The present invention was made in light of the above problem and has a purpose to provide a technique for simplifying pre-examination input tasks for a medical image imaging apparatus.

### Solution to Problem

In order to solve the above problem, the present invention includes a medical image imaging apparatus generating a medical image of an object, a position detection unit detecting a current position of the medical image imaging apparatus, an object candidate search unit searching object candidates to be examined based on the current position, a first operation unit receiving a selection operation of the object to be examined from among the object candidates, and an imaging condition setting unit setting imaging conditions of the object to be examined.

### Advantageous Effects of Invention

According to the present invention, a technique for simplifying pre-examination input tasks using an X-ray apparatus can be provided.

### BRIEF DESCRIPTION OF DRAWINGS

FIG. 1 is an explanatory diagram showing an overall configuration of an imaging support system.

FIG. 2 is a functional block diagram showing functions of a medical image imaging apparatus and an imaging support server.

FIG. 3 is an explanatory diagram showing examples of a search range and a screen display: (a) shows a search range; (b) shows a screen display example of a candidate bed number; and (c) shows an example of an object information input screen.

FIG. 4 is a flow chart showing a processing flow of an imaging support system related to the first embodiment.

FIG. 5 is an explanatory diagram showing an example of a hospital map.

FIG. 6 is an explanatory diagram showing an example of a position and a search range of a mobile X-ray apparatus.

FIG. 7 is an explanatory diagram showing examples of bed information and an object information management table: (a) shows the bed information; and (b) shows the object information management table.

FIG. 8 is an explanatory diagram showing a screen display example of the mobile X-ray apparatus 1.

FIG. 9 is a functional block diagram showing functions of a medical image imaging apparatus and an imaging support server.

FIG. 10 is a flow chart showing a processing flow of an imaging support system related to the second embodiment.

FIG. 11 is a functional block diagram showing functions of a mobile X-ray apparatus related to the third embodiment.

FIG. 12 is an explanatory diagram showing an overall configuration of an X-ray image diagnostic apparatus to be used for the fourth embodiment.

FIG. 13 is a functional block diagram showing a configuration of the operation console 210.

FIG. 14 is a flow chart showing a processing flow of an X-ray image diagnostic apparatus related to the fourth embodiment.

FIG. 15 is an explanatory diagram showing an example of regional information memorized in a regional information storage unit.

### DESCRIPTION OF EMBODIMENTS

An imaging support system related to the present invention is comprised of a medical image imaging apparatus generating a medical image of an object, a position detection unit detecting a current position of the medical image imaging apparatus, an object candidate search unit searching object candidates to be examined based on the current position, a first operation unit receiving an operation to select the object to be examined from among the object candidates, and an imaging condition setting unit setting imaging conditions of the object to be examined.

Also, an imaging support system related to the present invention may be further comprised of a display unit displaying the set imaging conditions and a second operation unit receiving an operation to change the displayed imaging conditions.

Also, the medical image imaging apparatus is a mobile apparatus moving in a facility, the position detection unit calculates a current position of the mobile apparatus in the facility, and the object candidate search unit may search the object candidates based on the current position in the facility.

Also, an imaging support system related to the present invention may further include a map information storage unit storing map information in which position information of each point in the facility corresponds to the layout of the facility. In this case, the position calculation unit may refer to the map information to calculate a current position in the facility.

Additionally, an imaging support system related to the present invention may further include a bed information storage unit storing bed information in which identification information of beds disposed in each point corresponds to position information of each point in the facility, a bed candidate search unit searching beds within a predetermined range including a current position in the facility by referring to the bed information to extract as bed candidates, and an object information storage unit storing object information in which identification information of objects who are using the respective beds corresponds to each of the identification information of the beds. In this case, the object candidate search unit may refer to the object information to extract identification information of the objects corresponding to identification information of the bed candidates extracted by the bed candidate search unit.

Also, a route storage unit storing a route where the mobile apparatus performs examination while moving in the facility may be further included. In this case, the bed candidate search unit may extract the beds on the route as the bed candidates.

Also, a route prediction unit predicting a route where the mobile apparatus performs examination while moving in the facility using the current position in the facility and the map information may be further included. In this case, the bed candidate search unit may extract the beds on the predicted route as the bed candidates.

Also, the medical image imaging apparatus may be connected to an imaging support server via a network. In this case, the medical image imaging apparatus may include a position signal transmission unit transmitting a signal showing a current position of the medical image imaging apparatus to the imaging support server, the first operation unit, and the imaging condition setting unit. Also, the imaging support server may include the position detection unit and the object candidate search unit. Then, the position detection unit may detect a current position of the medical image imaging apparatus based on a signal showing the current position transmitted from the position signal transmission unit.

Additionally, an imaging support system related to the present invention may further include a regional information storage unit storing regional information showing a standard body shape of people living in a country or a region where the medical image imaging apparatus is disposed. In this case, the position detection unit may include a region identification unit identifying a country or a region where the medical image imaging apparatus is disposed. Then, the imaging condition setting unit may refer to the regional information about a country or a region identified by the region identification unit to set imaging conditions based on a standard body shape of people living in the country or the region.

An imaging support system related to the present invention may further have a display unit displaying the set imaging conditions and a display control unit selecting a language used on a display screen of the display unit and displaying on the display screen. In this case, the regional information may further include language information used in the country or the region. Then, the display control unit, referring to the regional information about a country and a region identified by the region identification unit, may select and display a language to be used on the display screen based on language information used in the country and the region. Hereinafter, the embodiments of the present invention will be described in more detail using diagrams.

### First Embodiment

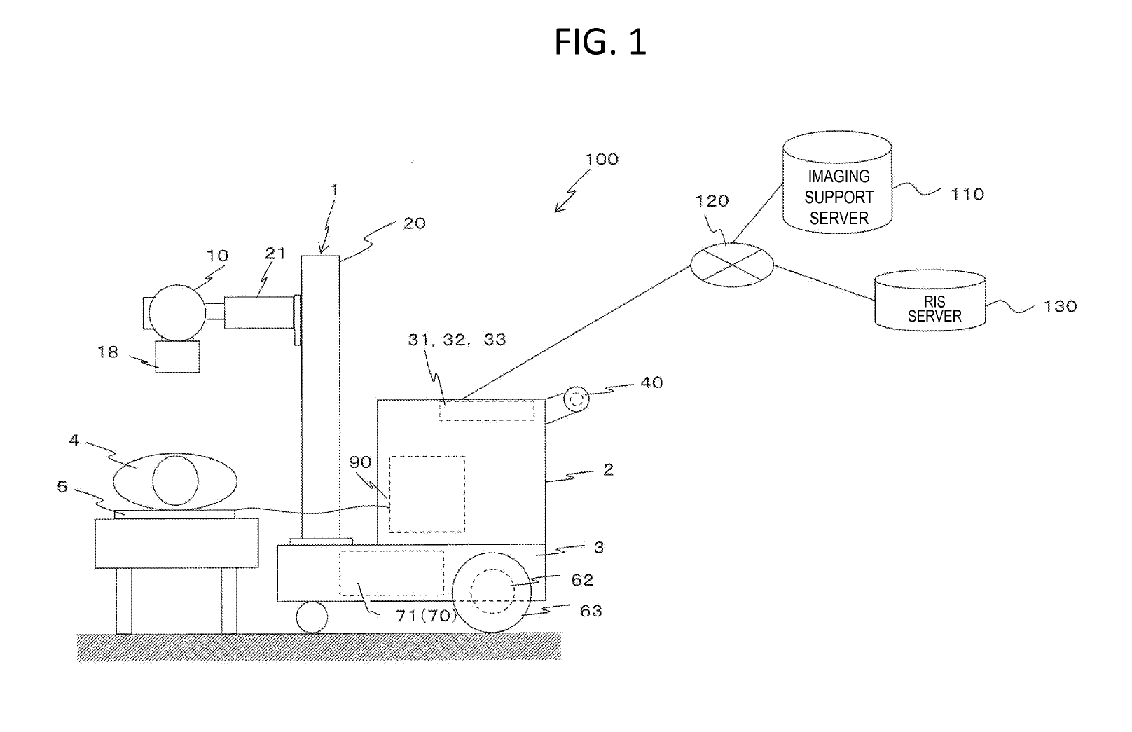

The first embodiment is an embodiment in which imaging conditions of an object is automatically input based on the position in a facility during a round visit using a mobile apparatus. In the first embodiment, the mobile X-ray apparatus 1 is used as an example of a medical image imaging apparatus, and a hospital is exemplified to describe a facility that uses the apparatus. Hereinafter, an imaging support system related to the first embodiment will be described based on FIGS. 1 to 8.

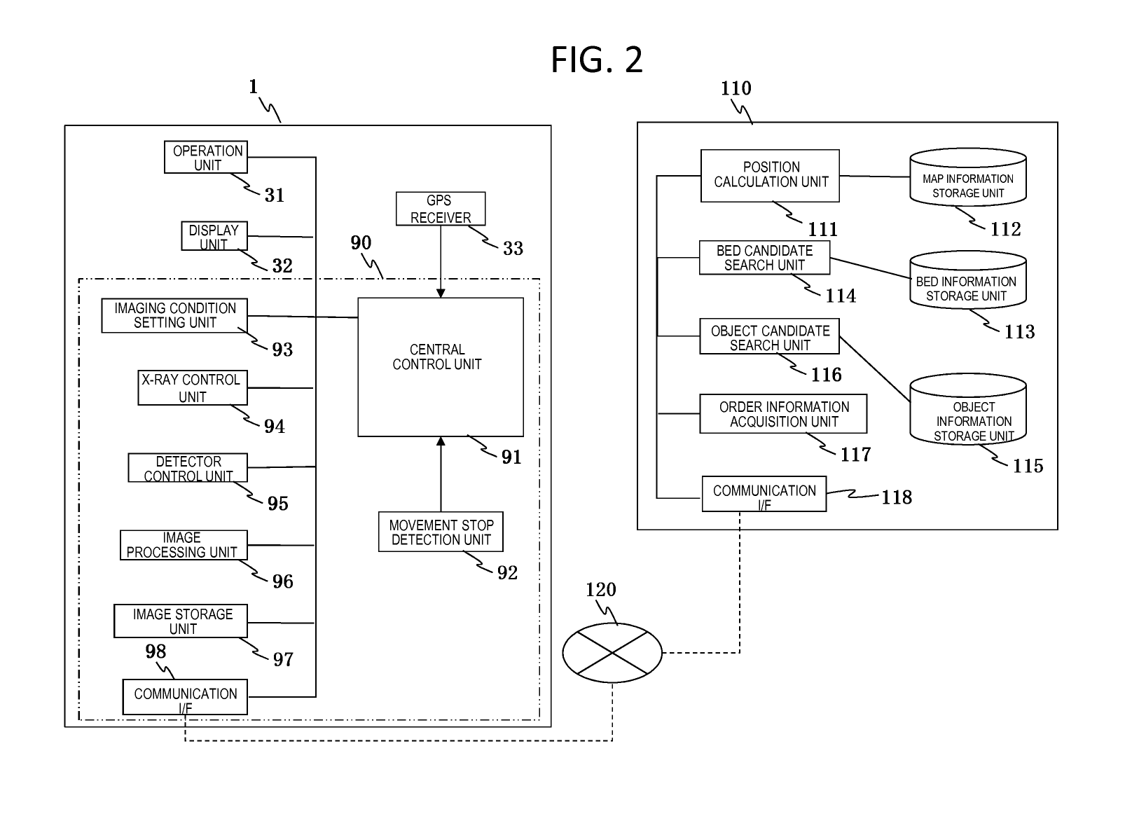

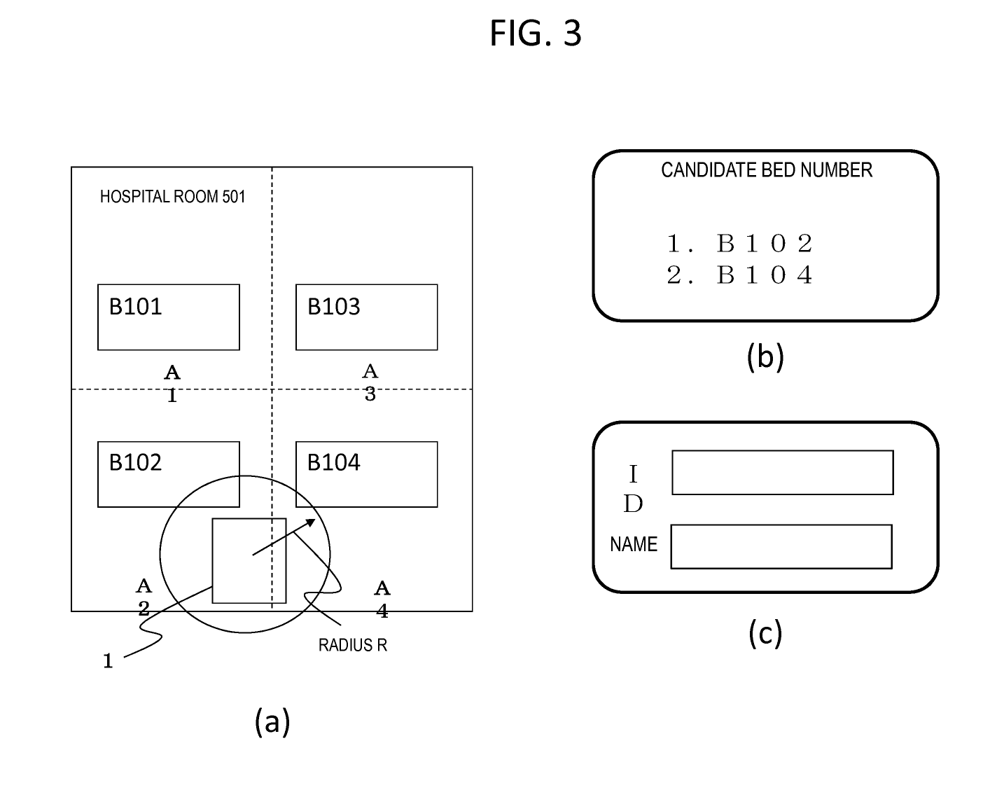

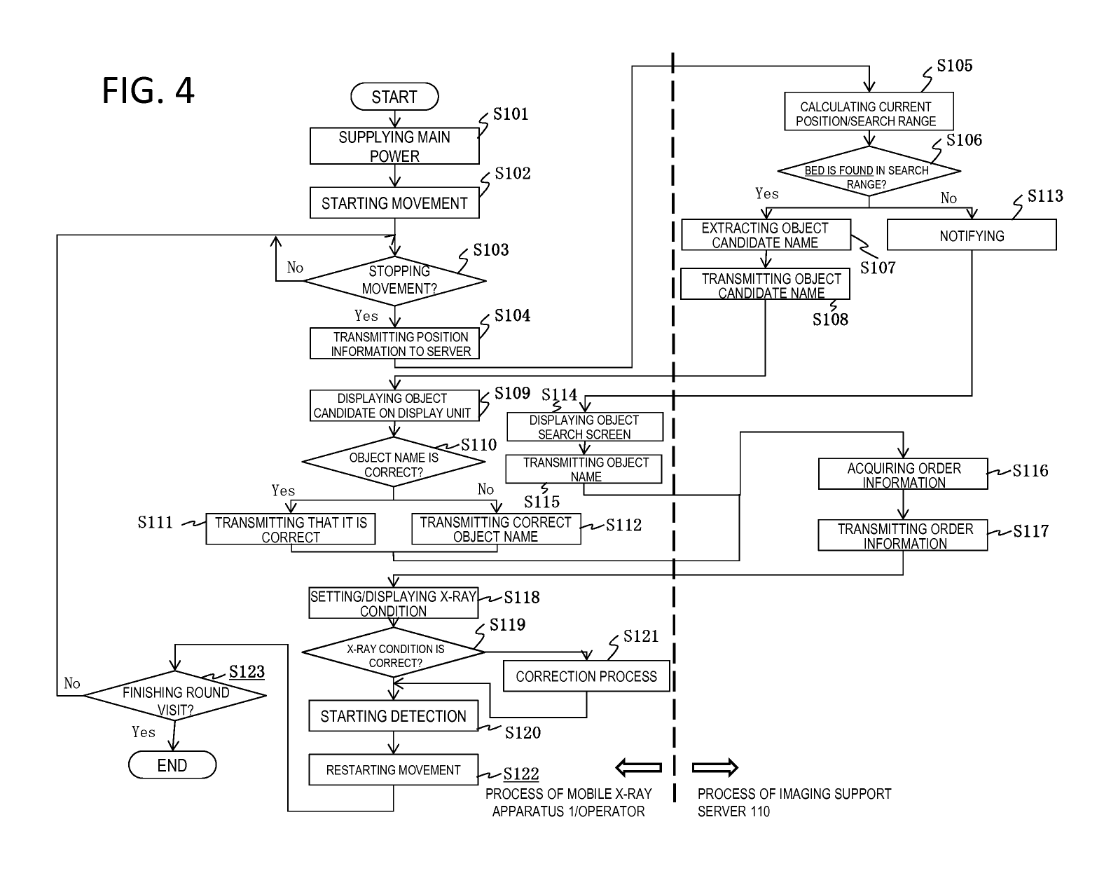

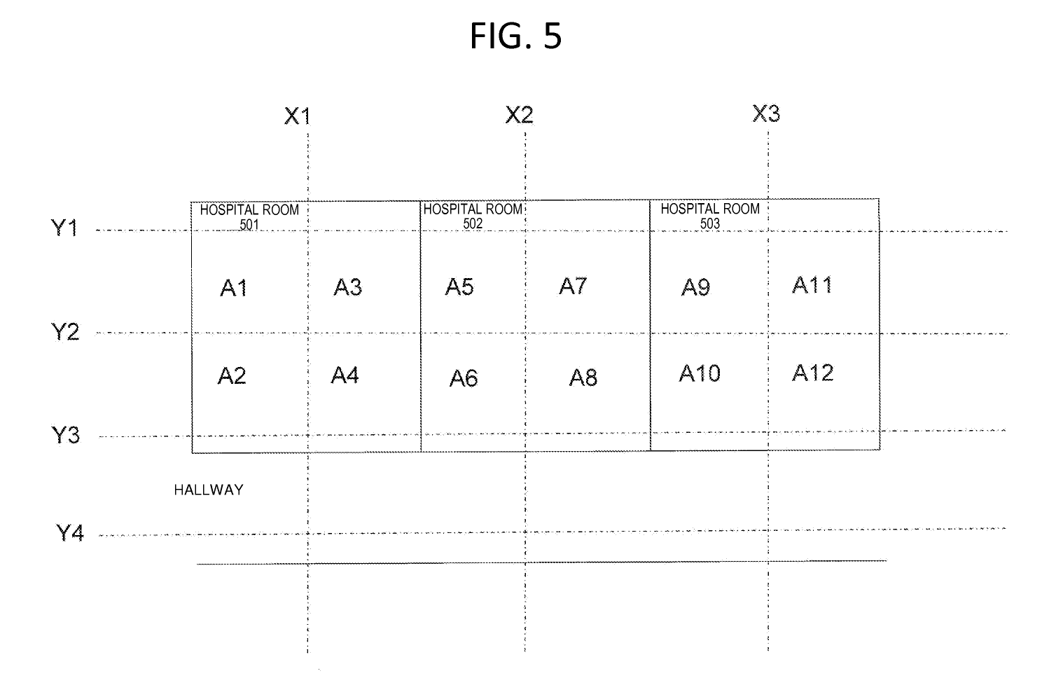

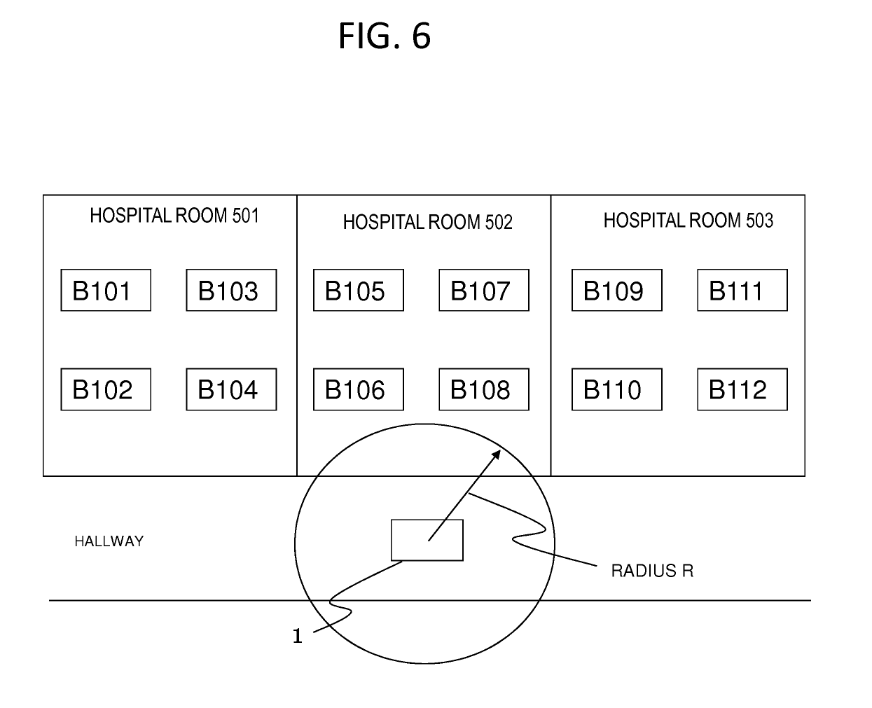

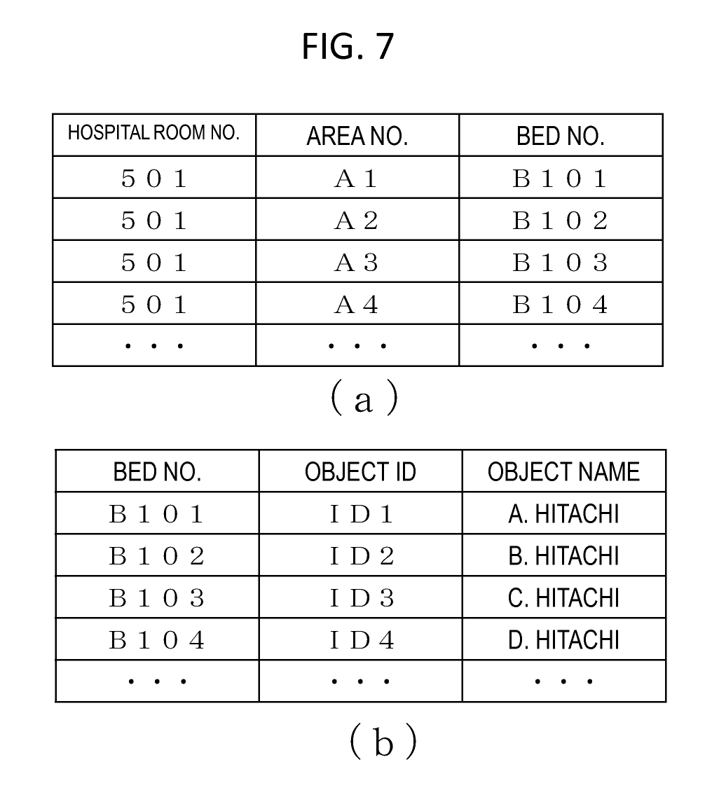

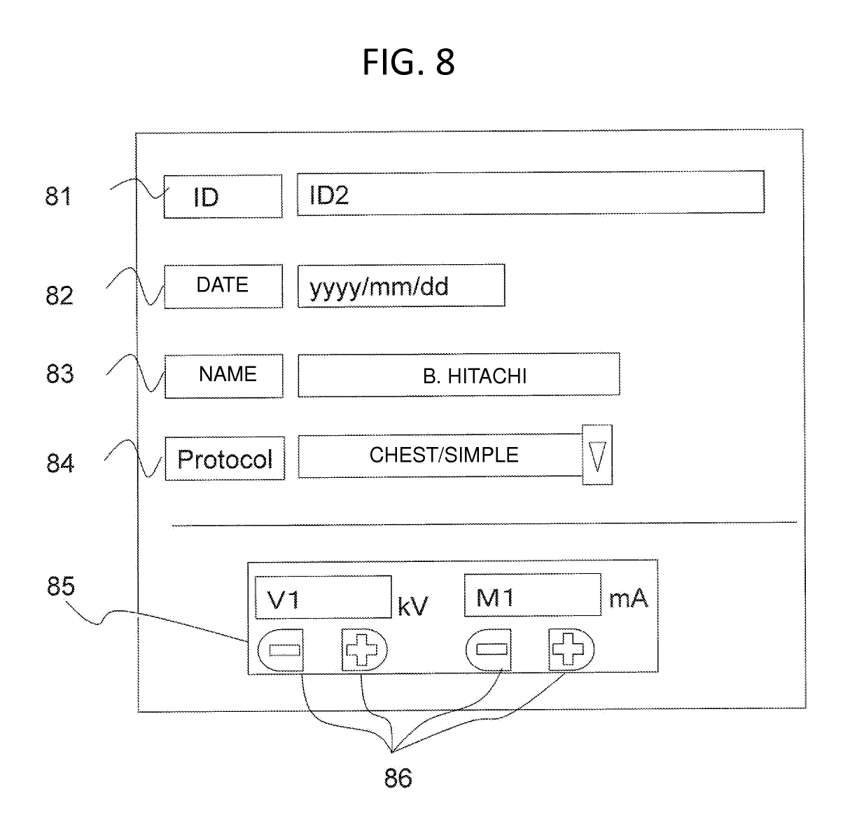

FIG. 1 is an explanatory diagram showing an overall configuration of an imaging support system. FIG. 2 is a functional block diagram showing functions of a medical image imaging apparatus and an imaging support server. FIG. 3 is an explanatory diagram showing examples of a search range and a screen display: (a) shows a search range; (b) shows a screen display example of a candidate bed number; and (c) shows an example of an object information input screen. FIG. 4 is a flow chart showing a processing flow of an imaging support system related to the first embodiment. FIG. 5 is an explanatory diagram showing an example of a hospital map. FIG. 6 is an explanatory diagram showing an example of a position and a search range of a mobile X-ray apparatus. FIG. 7 is an explanatory diagram showing examples of bed information and an object information management table: (a) shows the bed information; and (b) shows the object information management table. FIG. 8 is an explanatory diagram showing a screen display example of the mobile X-ray apparatus 1.

The imaging support system 100 shown in FIG. 1 includes the mobile X-ray apparatus 1 and the imaging support server 110. The mobile X-ray apparatus 1 and the imaging support server 110 are connected by wire or wireless communication via the network 120 (such as a LAN in a hospital). The network 120 is connected to the order information server 130 in which order information including an imaging procedure and imaging conditions of an object are stored. In the present embodiment, the RIS (Radio Information System) server 130 is exemplified to describe an order information server, and hereinafter, the order information server will be referred to as the RIS server. Without limiting to the mobile X-ray apparatus 1, the present invention can be applied also to a mobile medical image imaging apparatus such as an ultrasound diagnostic apparatus. Also, in case of radiation equipment such as an ultrasound diagnostic apparatus, the present invention can be applied by using the order information server 130 as a server that manages medical examination and treatment information by such an apparatus.

The mobile X-ray apparatus 1 mainly has the main body 2 and the traveling carriage 3 on which the main body 2 is mounted. The traveling carriage 3 has the running motor 62 and the wheels 63 and runs by driving the wheels 63 by the motor. The support column 20 is mounted on the front side of the traveling carriage 3. On the upper side of the support column 20, the arm 21 comprised of a pantographic arm and telescopic arm is mounted. On the open end side of the arm 21, the X-ray generation unit 10 generating an X-ray and the diaphragm device 18 restricting an X-ray irradiation range are mounted. Positions of the X-ray generation unit 10 and the diaphragm device 18 can be moved so that an X-ray is irradiated to an imaging site of an object by expanding, contracting, and rotating the arm 21. Also, specifying the longitudinal direction of the support column 20 as a rotation axis, the arm 21 can be rotated in a circumferential direction.

Additionally, the main body 2 has the operation unit 31 including a mouse, a keyboard, and a trackball as well as the display unit 32 including a display panel comprised using a liquid crystal monitor etc. Also, inside the main body 2, the control device 90 controlling operation of the mobile X-ray apparatus 1 is installed.

When examination is performed, the X-ray detector 5 detecting an X-ray transmitted through the object 4 is mounted in the opposite position to the X-ray generation unit 10 across the object 4. In the present embodiment, an FPD (Flat Panel Detector) is mounted as the X-ray detector 5. The FPD is an example of an X-ray detector, and any devices such as an imaging plate, an X-ray film, etc can be used for generating a roentgenogram or an X-ray image of an object by detecting a transmitted X-ray. Also, although the FPD may be the one connected to the main body 2 of the mobile X-ray apparatus 1 by wire or wirelessly as well as a so-called portable FPD configured differently from the mobile X-ray apparatus 1, the FPD that was connected to the control device 90 by wire is exemplified for description in the present embodiment.

Inside the traveling carriage 3, the power unit 70 including the battery 71 to supply electricity to the respective components of the mobile X-ray apparatus 1 is provided.

On the opposite side to the support column 20 on the main unit 2 (near the rear upper surface), the traveling handle 40 that an operator holds to travel the mobile X-ray apparatus 1 is provided. Although the traveling handle 40 is omitted to be shown in the diagram, an electromagnetic brake is provided. When an operator holds the traveling handle 40, the electromagnetic brake is released, which can travel the mobile X-ray apparatus 1. When an operator releases hands from the traveling handle 40, the electromagnetic brake activates, which can brake the mobile X-ray apparatus 1.

As shown in FIG. 2, the mobile X-ray apparatus 1 includes the operation unit 31, the display unit 32, the GPS receiver 33 receiving a GPS (Global Positioning System) signal, and the control device 90. The control device 90 is comprised of the central control unit 91 controlling various operations, the movement stop detection unit 92 detecting that the mobile X-ray apparatus 1 stopped, the imaging condition setting unit 93 setting imaging conditions (equivalent to X-ray irradiation conditions such as a tube voltage and a tube current), the X-ray control unit 94 controlling operation of the X-ray generation unit 10, the detector control unit 95 performing a reading operation of a transmitted X-ray signal generated by the X-ray detector 5, the image processing unit 96 generating an X-ray image of the object 4 based on the transmitted X-ray signal, the image storage unit 97 storing X-ray images, and the communication interface (communication I/F) 98 to perform communication between the mobile X-ray apparatus 1 and external devices.

The central control unit 91 is electrically connected to each of the operation unit 31, the display unit 32, the GPS receiver 33, the movement stop detection unit 92, the X-ray control unit 94, the detector control unit 95, the image processing unit 96, the image storage unit 97, and the communication I/F 98. Also, the mobile X-ray apparatus 1 is connected to the network 120 via the communication I/F 98.

In the present embodiment, a GPS receiver is to be used as a position detecting device, and the GPS receiver 33 is provided for the mobile X-ray apparatus 1. The GPS receiver 33 receives a GPS signal of three or more points and calculates a longitude, latitude, and altitude, and it is desirable that positions on a horizontal surface and vertical surface in a hospital can be detected.

On the other hand, the imaging support server 110 is comprised of the position calculation unit 111 calculating a current position of the mobile X-ray apparatus 1 in a hospital, the map information storage unit 112 storing map information corresponding to position information of each point in the hospital layout, the bed information storage unit 113 storing bed information in which position information of each point in the hospital corresponds to identification information of beds disposed at each point, the bed candidate search unit 114 searching beds within a predetermined range including a current position by referring to the bed information to extract as a bed candidate, the object information storage unit 115 storing object information in which identification information of each bed corresponds to identification information of objects who are using each bed, the object candidate search unit 116 searching an object candidate to be examined based on a current position, the order information acquisition unit 117 acquiring order information including information about examinations to be performed for each object such as an imaging site, an imaging procedure, X-ray conditions, etc., and the communication interface (communication I/F) 118 to perform communication between the imaging support server and external devices. The mobile X-ray apparatus 1 is connected to the network 120 via the communication I/F 118.

The above “identification information of beds” is not to individually identify the respective beds but to identify a position where a bed is disposed in a hospital. Therefore, for example, in a case where a bed is removed to dispose a new bed in the same position for cleaning, the same bed number as the bed disposed in the position before cleaning is provided for the new bed.

For example, as shown in FIG. 3(a), the hospital room 501 is divided into four regions A1 to A4 so that it is previously determined that they are referred to as: the number of the bed located in A1: B101, the number of the bed located in A2: B102, the number of the bed located in A3: B103, and the number of the bed located in A4: B104. There is a case where the bed of the bed number B101 located in A1 was moved to A2 after cleaning or rearrangement of the bed. In this case, the bed number of this bed is changed to B102. Thus, in the present embodiment, a bed number corresponds to a position in a hospital at the 1:1 ratio.

In the present embodiment, although a position detecting device detecting a position of the mobile X-ray apparatus 1 is configured using the GPS receiver 33 and the position detection unit 111, a position detection device is not limited to this configuration. For example, it may be configured so that a position of the mobile X-ray apparatus 1 is detected according to which wireless receiver (wireless transmitter) provided in each position was used after providing a wireless transmitter (or wireless receiver) for the mobile X-ray apparatus 1 and providing wireless receivers (or wireless transmitters) for the respective points in a hospital to perform wireless communication between these wireless transmitters and wireless receivers.

As an example of the above wireless transmitter/receiver, RFID (Radio Frequency Identification) is used to provide an RF reader for the mobile X-ray apparatus 1 and provide RFID tags in the respective points in a hospital. Then, it may be configured so that the RFID tags are read while the mobile X-ray apparatus 1 are moving to calculate a current position of the mobile X-ray apparatus 1 based on information of the RFID tags. On the contrary to the above example, although an RFID tag and RF readers may be provided respectively for the mobile X-ray apparatus 1 and the respective points in the hospital, it is more desirable to provide RFID tags for the respective points in the hospital in light of the cost because an RFID tag is generally cheaper than an RF reader. Also, a position detection device for which an operator inputs a bed number and a hospital room number may be provided.

A processing flow related to the first embodiment will be described along the respective steps of FIG. 4.

(Steps S101 to S103)

In order to start a round visit, the main power of the mobile X-ray apparatus 1 is turned on (S101). An operator holds the traveling handle 40 and moves the mobile X-ray apparatus 1 while releasing the electromagnetic brake that is not shown in the diagram, and the mobile X-ray apparatus 1 starts movement (S102). The movement stop detection unit 92 determines whether the movement of the mobile X-ray apparatus 1 is stopped or not (S103). If it is affirmative, the flow proceeds to Step S104, and if it is negative, the flow goes back to Step S103.

(Step S104)

When the stop is detected, the GPS receiver 33 receives a GPS signal, and the central control unit 91 transmits it to the server 110 via the communication I/F 98. That is, the central control unit 91 controls transmission of the GPS signal.

(Step S105)

The position calculation unit 111 receives a GPS signal via the communication I/F 118. Then, referring to map information stored in the map information storage unit 112, a current position of the mobile X-ray apparatus 1 and a search range centering on the current position are calculated (S105).

The position calculation unit 111 calculates coordinates (X, Y, Z) of a current position of the mobile X-ray apparatus 1 based on a received GPS signal. Next, as shown in FIG. 3(a), coordinates of a search range of a radius R centering on a current position are calculated. The radius R value may be changed as needed. For example, in a case where a distance between beds and the respective divided regions are relatively small, the radius R value may be relatively reduced, and in a case where a distance between beds and the respective divided regions are relatively large, the radius R value may be relatively increased. Additionally, in FIG. 3(a), although a circular search range is drawn, a shape of a search range may be a fan shape centering on the mobile X-ray apparatus 1, and the shape is not limited to a circle.

Then, referring to the map information of FIG. 5, a candidate area included within a search range of a radius R is calculated. In the map information of FIG. 5, the layout of a hospital corresponds to coordinates (X, Y) of each point in the hospital. Actually, although a Z coordinate showing a height direction (floor number) of the hospital is included, the same floor map of the hospital is shown, and the Z coordinate is omitted in FIG. 5 for convenience of description.

In an example of FIG. 3(a), the divided regions A2 and A4 are included within a search range centering on a current position, and these two divided regions are extracted as candidate areas. As shown in FIG. 6, in a case where the mobile X-ray apparatus 1 is in a hallway, the number of a closest hospital room may be calculated as a candidate area.

(Step S106)

The bed candidate search unit 114 refers to bed information stored in the bed information storage unit 113 to search a bed within a candidate area calculated in Step S105 (S106).

A bed searched as a result of the present step becomes a bed candidate to be used by an object to be examined. The bed information of FIG. 7(a) is formed by corresponding to hospital room numbers, area numbers, and bed numbers. A data structure of the bed information is just an example, and for example, coordinates of each position in a hospital may be directly associated with the bed numbers. The bed candidate search unit 114 searches a bed number corresponding to each divided region in a case where a candidate area for each divided region in Step S105. Also, all the bed numbers in a hospital room are searched in a case where the hospital room number is searched as a candidate area. In a case where a plurality of bed numbers are identified, they are prioritized in order from the number of a closer bed.

As a result of the search in the present step, if there is a bed within a search range, the flow proceeds to Step S107, and if not, the flow proceeds to Step S113.

(Steps S107 and 108)

The object candidate search unit 116 extracts an object name (S107) corresponding to a bed number searched in Step S106 from among object information stored in the object information storage unit 115 to transmit the object name to the mobile X-ray apparatus 1 (S108). An object name extracted in Step S107 becomes an object candidate. The object information of FIG. 7(b) is formed by corresponding to bed numbers, ID information that can identify an object individually, and object names. In a case where a plurality of bed numbers are searched in Step S106, an object name corresponding to each bed is identified.

(Step S109)

The mobile X-ray apparatus 1 displays received object candidate names on the display unit 32 (S109). If there is a plurality of object names, they are prioritized in the order of proximity to the mobile X-ray apparatus 1 before the transmission and are arranged and displayed along the order as shown in FIG. 3(b) for example.

(Steps S110 to S112)

An operator checks a displayed object name (S110). If it is correct, this is input from the operation unit 31 and transmitted to the imaging support server 110 (S111). If it is wrong or if a plurality of object names are displayed, an object name to be examined is input/selected and transmitted to the server (S112). Then, the flow proceeds to Step S116.

(Steps S113 to S115)

If it is determined that there is no bed in Step S105, the bed candidate search unit 114 notifies the mobile X-ray apparatus 1 of that there is no candidate bed (S113). If the central control unit 91 is notified of that there is no candidate, a screen for an operator to input an object name, such as a screen where at least either one of object IDs that can individually identify an object name or an object can be input as shown in FIG. 3(c) for example, is displayed on the display unit 32 (S114). An operator inputs an object name from the operation unit 31. The central control unit 91 transmits an input object name to the imaging support server 110 (S115). Then, the flow proceeds to Step S116.

(Steps S116 and S117)

The order information acquisition unit 117 acquires order information corresponding to an object name identified in S111, S112, or S113 from the RIS server 130 (S116) and transmits it to the mobile X-ray apparatus 1 (S117). The mentioned order information includes X-ray conditions (for example, mA, kV, mAs, etc.) corresponding to an examination procedure to be received by the object.

(Step S118)

The imaging condition setting unit 93 sets X-ray conditions included in received order information, and the display unit 32 displays the setting values (S118). Ina case where an object name identified in S111, S112, or S113 is “B. HITACHI”, the order information acquisition unit 117 acquires and displays order information of “B. HITACHI”.

FIG. 8 shows a display example of the display unit 32 at the time when the present step ends. The FIG. 8 screen includes the “ID” field 81, the “Date” field 82, the “Name” field 83, the “Protocol” field 84, the “X-ray condition display” field 85, and the adjustment buttons 86. An example of FIG. 8 displays a case where “Protocol” (examination procedure) is routine chest imaging; and X-ray conditions include a tube voltage V1 and a tube current M1. By operating the operation buttons 86 to increase or decrease automatically input setting values of a tube voltage and a tube current, X-ray conditions can be finely adjusted.

(Steps S119 to S121)

An operator visually checks displayed X-ray information (S119) and presses down an X-ray exposure button if the information is correct to execute an examination (S120). According to the set X-ray conditions, the X-ray control unit 94 irradiates an X-ray, and then the detector control unit 95 reads out a transmission X-ray signal from FPD. The image processing unit 96 generates an X-ray image of an object based on the transmission X-ray signal, and the image storage unit 97 stores the image. The X-ray image may be displayed on the display unit 32. If displayed X-ray conditions are wrong, correction processing is performed (S121), and then the examination starts (S120). The mentioned correction processing includes a case where the input screen of object information shown in FIG. 3(c) is displayed again to acquire, set, and display order information after a correct object name is input, in addition to the fine adjustment of the X-ray conditions.

After the examination ends, the mobile X-ray apparatus 1 resume the movement (S122). If there is no subsequent examination, the mobile X-ray apparatus 1 is brought back to a storage position, the main power is turned off, and then a series of processes end. If there is a subsequent examination, the flow goes back to Step S103, and then the processes after Step S103 are performed again (S123).

According to the present embodiment, because order information of an object to be examined can be automatically input based on a position of the mobile X-ray apparatus 1 in a hospital, time and effort for an operator to input the order information can be reduced. Hence, an operator's workload is reduced, which also can prevent erroneous input of the order information.

Although processes such as calculating a current position is started by specifying that the movement stop detection unit 92 detects the movement stop of the mobile X-ray apparatus 1 as a trigger in the above description, a time elapsed after detecting the movement stop (an electromagnetic brake is ON) is measured, and then the fact that a predetermined time elapsed may be specified as a trigger.

Also, the mobile X-ray apparatus 1 has an object information acquisition button, and it may be configured so that pressing down this button by an operator is a trigger to start processes after Step S104 of FIG. 4. Also, when a process starting area is previously described in map information of a hospital; and the position calculation unit 111 calculates that the mobile X-ray apparatus 1 entered into the process starting area, it may be configured so that this is specified as a trigger to display an object candidate on the display unit 32.

### Second Embodiment

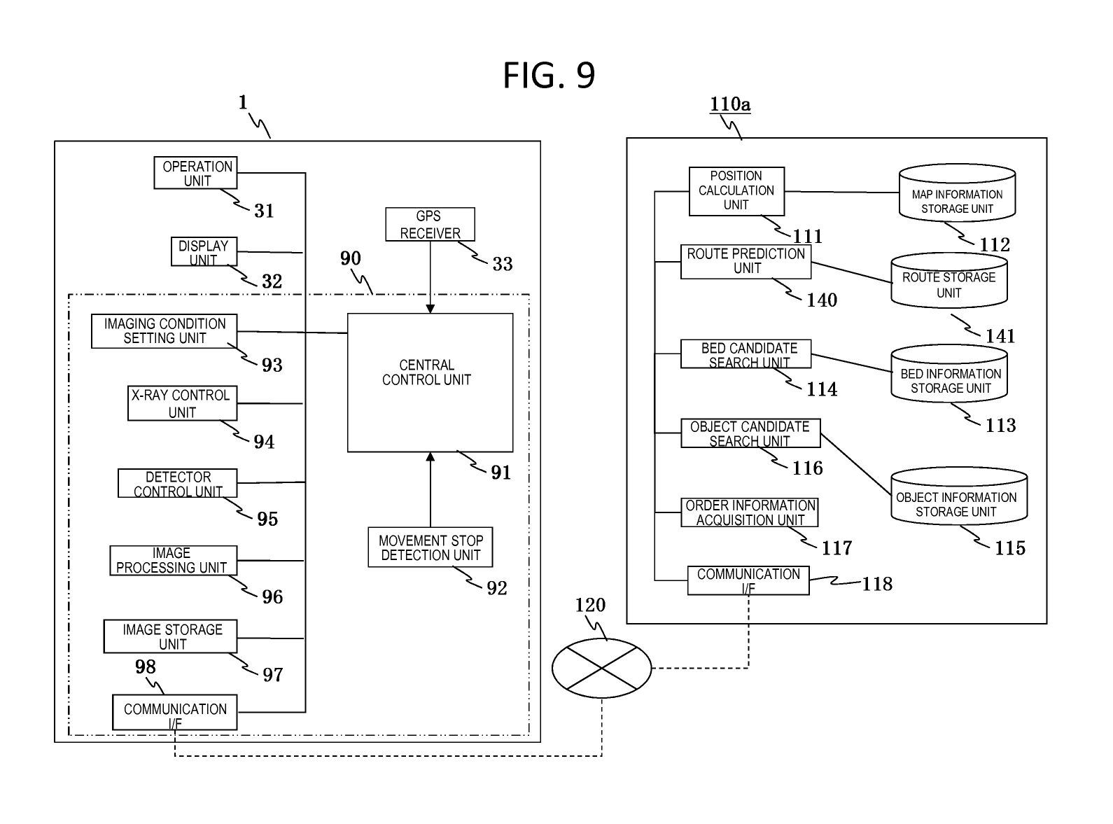

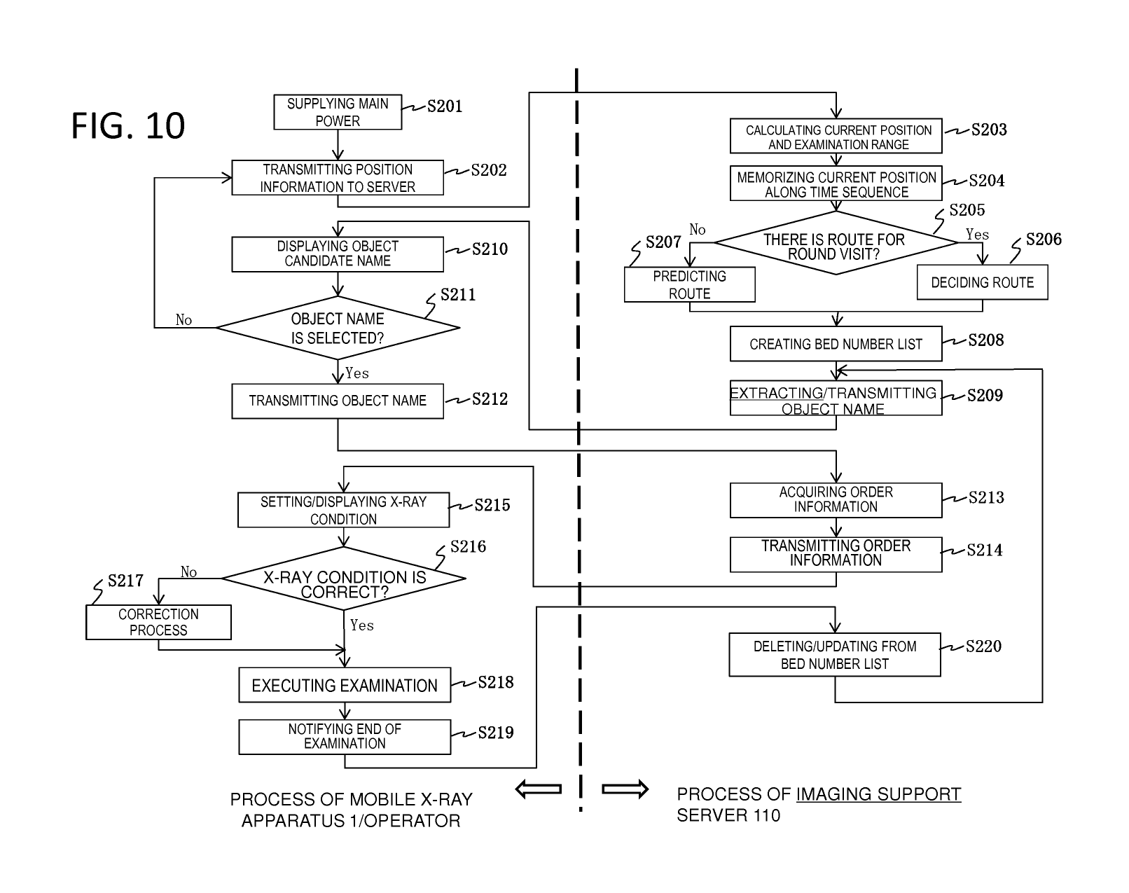

The second embodiment is an embodiment to memorize a route for a round visit and search an object candidate along the route. Hereinafter, the second embodiment will be described using FIGS. 9 and 10. FIG. 9 is a functional block diagram showing functions of a medical image imaging apparatus and an imaging support server. FIG. 10 is a flow chart showing a processing flow of an imaging support system related to the second embodiment.

The imaging support server 110a has the route prediction unit 140 predicting a route for a round visit and the route storage unit 141 storing the route, in addition to the configuration of the imaging support server 110 of the first embodiment. The other configurations are the same as the imaging server 110, and the descriptions will be omitted. Also, a medical image imaging apparatus that is similar to the mobile X-ray apparatus 1 used in the first embodiment will be used, and the description will be omitted. Hereinafter, the flow of the present embodiment will be described along the respective steps of FIG. 10.

(Steps S201 and S202)

The main power of the mobile X-ray apparatus 1 is turned on (S201). After turning on the main power, the GPS receiver 33 starts receiving a GPS signal and transmits it to the server 110a (S202). Also, after turning on the main power, an operator moves the mobile X-ray apparatus 1.

(Step S203)

The position calculation unit 111 calculates a current position of the mobile X-ray apparatus 1 (S203).

(Step S204)

The route storage unit 141 stores a current position calculated in Step S203 along the time series (S204). A trajectory of current positions along the time series is stored as route information. If the route information is stored in addition to a position where an examination is executed, an order for executing the examination along the route can be managed.

(Steps S205 and S206)

The route prediction unit 140 refers to route information of the route storage unit 141 to determine whether there is a current position on a route for a round visit or not (S205). If so, the route for a round visit is decided as a predicted route (S206), and then the flow proceeds to Step S208. If not, the flow proceeds to Step S207.

(Step S207)

If no route is found, the route prediction unit 140 refers to map information stored in the map information storage unit 112 and predicts a route for a round visit (S207). Predicting a route for a round visit may be performed by, for example, checking a trajectory after turning on the main power in S201 with a hospital map of the same floor, calculating a vector showing a moving direction of the mobile X-ray apparatus 1, and arranging hospital rooms and divided regions located in the moving direction in order from a relatively close position to the mobile X-ray apparatus 1.

(Step S208)

The bed candidate search unit 114 searches a bed candidate on a route for a round visit to refer bed information stored in the bed information storage unit 113. Then, bed numbers of each bed candidate are extracted to make a list of bed numbers arranged in the order of the route for a round visit (S208). “The order of the route for a round visit” is not necessarily limited to the order close to the mobile X-ray apparatus 1. For an example of a route for a round visit determined in Step S206, in a case where a round visit was previously performed in order from a farther position than a current position of the mobile X-ray apparatus 1, a bed number list is made, in which bed numbers on a route for a round visit is described in order from a relatively farther bed. On the other hand, in case of a route for a round visit predicted in Step S207, because this is a route for a round visit that is newly predicted this time, a bed number list may be made, in which hospital rooms and divided regions located in a moving direction are arranged in order from the one relatively close to a current position of the mobile X-ray apparatus 1. An algorithm to make a bed number list may be configured so that the ascending order, descending order, etc. can be set accordingly using a setting screen provided separately (omitted to be shown in the diagram).

(Step S209)

The object candidate search unit 116 refers object information stored in the object information storage unit 115, extracts object candidate names corresponding to a bed number described in a bed number list, and then transmits it to the mobile X-ray apparatus 1 (S209).

(Steps S210 and S211)

Object candidate names are displayed in the order described in the bed number list on the display unit 32 of the mobile X-ray apparatus 1 (S210). The flow proceeds to Step S212 when an operator selects a name of an object to be examined, and the flow goes back to Step S202 to perform processes of Steps S202 to S211 repeatedly, i.e., to execute loop processing when the operator does not select a name. In Step S205 during the second and subsequent loop processing, whether the apparatus moves along a route for a round visit predicted or determined in the previous loop processing or not. In a case where the movement is along the route for a round visit after the determination, the flow proceeds to Step S206. On the other hand, in a case where the movement is not along the route for a round visit, the flow proceeds to Step S207, and the route prediction unit 140 predicts a new route for a round visit based on current position information. Hence, current position calculation and prediction/determination of a route for a round visit are performed again, and object candidate names are updated and displayed according to the current position change.

(Step S212)

The central control unit 91 transmits selected object names to the imaging support server 110 (S212).

(Steps S213 and S214)

The order information acquisition unit 117 acquires order information of received object names from the RIS server 130 (S213) and transmits it to the mobile X-ray apparatus 1 (S214).

(Steps S215 to S217)

The imaging condition setting unit 93 sets X-ray conditions according to the received order information and displays the setting values on the display unit 32 (S215). An operator checks whether the displayed contents are correct (S216). If the displayed contents are correct, the flow proceeds to Step S218. If the displayed contents are wrong, correction processing is performed for X-ray information (S217). The correction processing mentioned here includes manual re-inputting object names, transmitting them to the imaging support server 110a, re-acquiring order information, and increasing and decreasing X-ray conditions to perform fine adjustment. Then, the flow proceeds to Step S218.

(Steps S218 and S219)

An operator executes an examination (S218). When the examination completes the central control unit 91 performs notification of examination completion for the imaging support server 110a (S219).

The bed candidate search unit 114 makes a new bed number list in which bed numbers of objects that had been examined was deleted from the bed number list made in S208 (S220). The processes from Step S201 to Step S220 will be repeatedly executed until the main power is turned OFF.

According to the present embodiment, because a route for a round visit is predicted, and objects that can be examined are searched and displayed at any time when a round visit starts, the objects can be displayed with the high priority when the round visit actually reaches the vicinity of the objects to be examined. Then, selecting the object can automatically input order information, which can further shorten preparation time required to start the examination after the round visit reaches the vicinity of the object.

### Third Embodiment

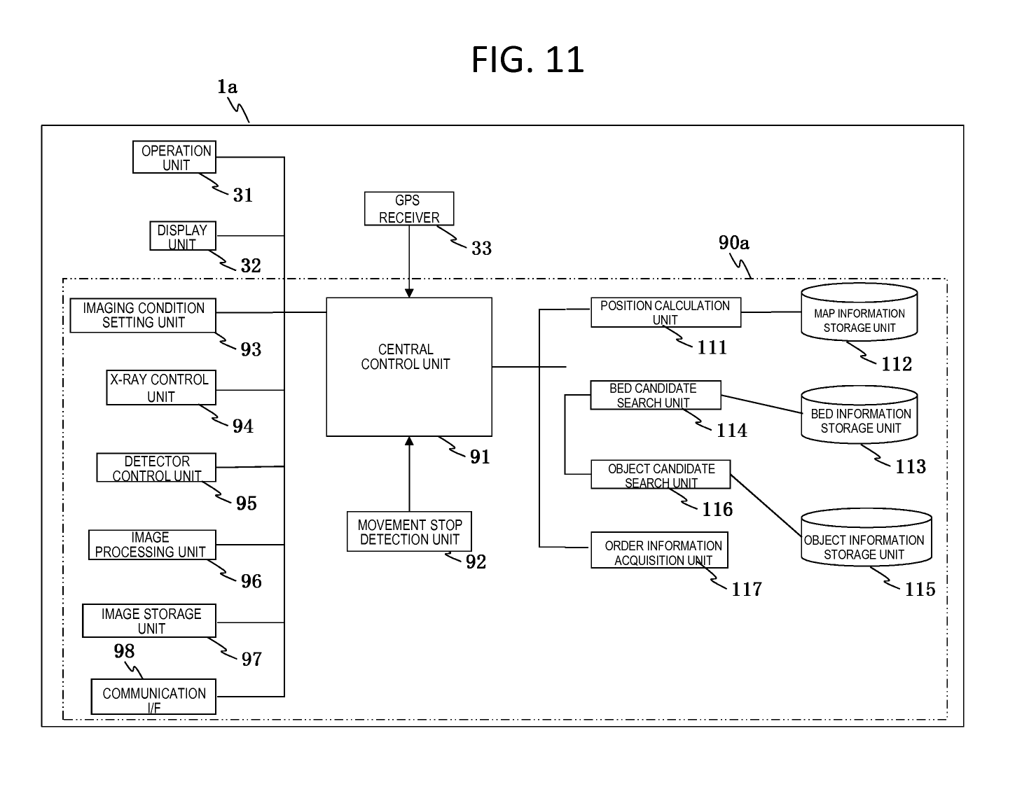

The third embodiment is an embodiment in which the mobile X-ray apparatus 1 calculates a current position and regional information as well as identifies bed numbers and object names. Hereinafter, the third embodiment will be described using FIG. 11. FIG. 11 is a functional block diagram showing functions of a mobile X-ray apparatus related to the third embodiment.

As shown in FIG. 11, the mobile X-ray apparatus 1a related to the third embodiment further includes the position calculation unit 111, the map information storage unit 112, the bed information storage unit 113, the bed candidate search unit 114, the object information storage unit 115, the object candidate search unit 116, and the order information acquisition unit 117 in addition to the configuration of the control device 90 of the first embodiment. It is desired that the mobile X-ray apparatus 1 is small and light, and mountable hardware can be restricted. In that case, it may be configured so that requests for downsizing and weight reduction of the hardware can be met by storing a hospital map of a floor where the mobile X-ray apparatus 1 is used and only information of objects who are hospitalized on the floor in each storage unit of the mobile X-ray apparatus 1.

In the mobile X-ray apparatus 1a, the GPS receiver 33 receives a GPS signal when a round visit starts. The position calculation unit 111 calculates a current position and a search range based on the GPS signal. Then, map information stored in the map information storage unit 112 is calculated to calculate a candidate area. The bed candidate search unit 114 refers to bed information stored in the bed information storage unit 113 to identify bed numbers in a candidate area. The object candidate search unit 116 refers to the object information storage unit 115, extracts object candidate names corresponding to bed numbers in a candidate area, and then displays them on the display unit 32.

When an operator selects an object displayed on the display unit 32, the order information acquisition unit 117 acquires order information from the RIS server 130 via the network 120. The imaging condition setting unit 93 sets X-ray conditions based on acquired order information, and the setting values are displayed on the display unit 32.

According to the present embodiment, network connection is not required during processes from receiving a GPS signal to identifying an object, which contributes to alleviate network congestion, and accelerating processes until order information is automatically input can be expected.

In the above description, although an example where the mobile X-ray apparatus 1 sets X-ray conditions automatically alone based on the mobile X-ray apparatus 1 related to the first embodiment is described, it may be configured so that the mobile X-ray apparatus 1 can execute a search for a route of a round visit alone by mounting the route prediction unit 140 and the route storage unit 141 on the mobile X-ray apparatus 1a without connecting to a network.

### Fourth Embodiment

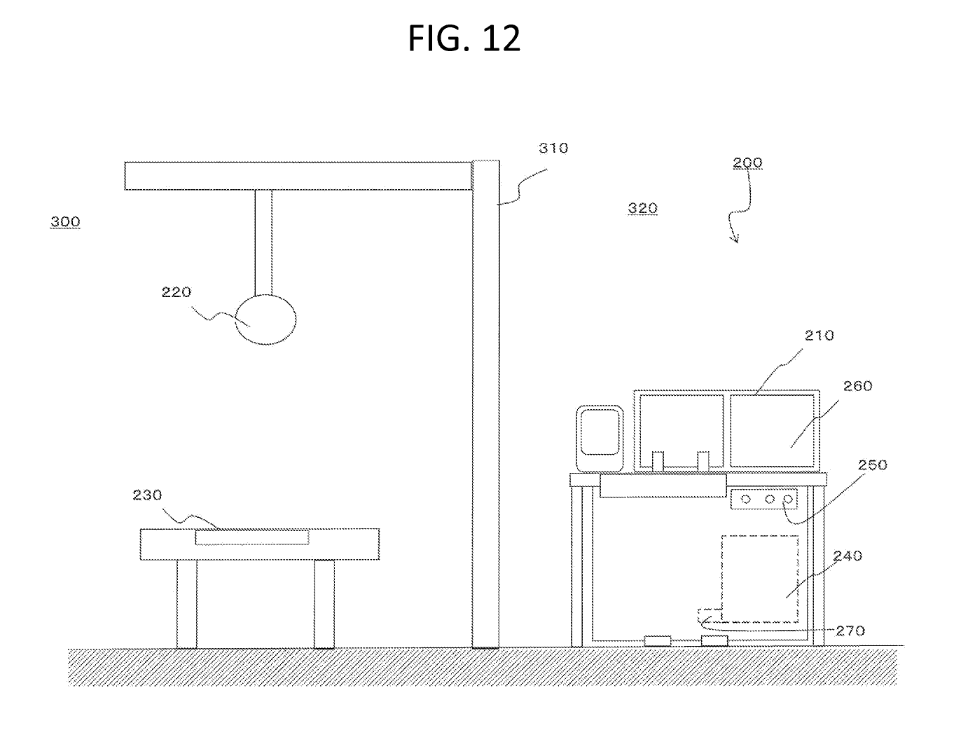

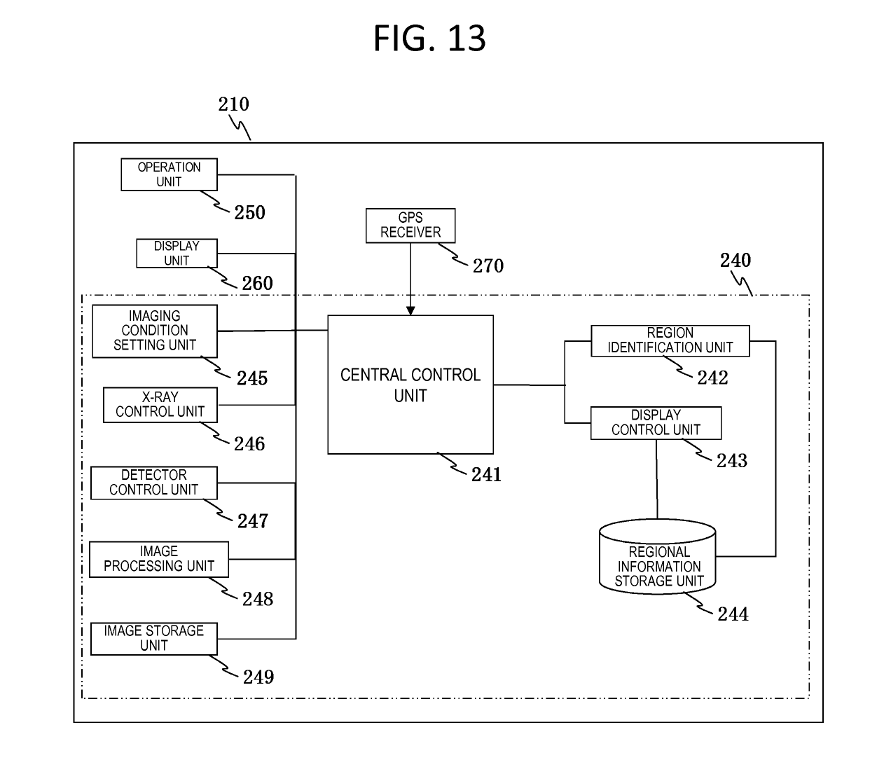

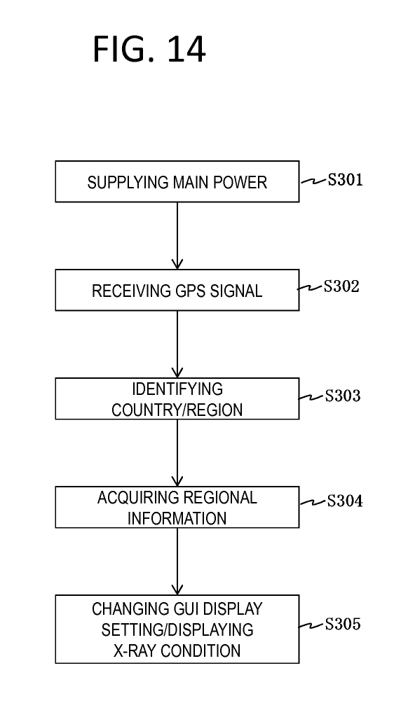

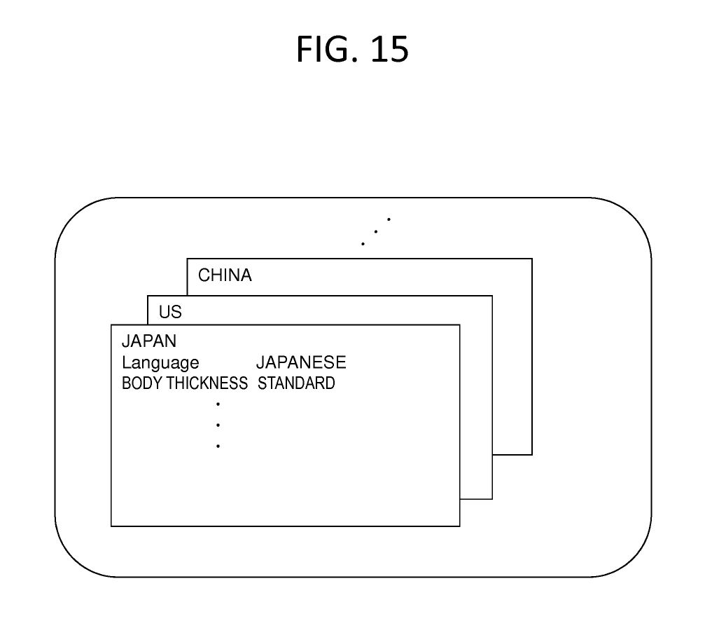

The fourth embodiment is an embodiment in which a language to be used for a GUI (Graphical User Interface) and X-ray conditions suitable for physique of people residing in a region where the mobile X-ray apparatus 1 is used are automatically input. Additionally, the present embodiment can be combined with any of the first embodiment to the third embodiment. Hereinafter, FIGS. 12 to 15 are used for the description. FIG. 12 is an explanatory diagram showing an overall configuration of an X-ray image diagnostic apparatus to be used for the fourth embodiment. FIG. 13 is a functional block diagram showing a configuration of the operation console 210. FIG. 14 is a flow chart showing a processing flow of an X-ray image diagnostic apparatus related to the fourth embodiment. FIG. 15 is an explanatory diagram showing an example of regional information memorized in an regional information storage unit.

FIG. 12 shows the stationary X-ray image diagnostic apparatus 200. The X-ray image diagnostic apparatus 200 includes the X-ray generation device 220 and the X-ray detector 230 installed in the imaging chamber 300 and the operation console 210 installed in the operation chamber 320 adjacent to the imaging chamber 300 across the partition 310.

The X-ray generation device 220 may use an overhead traveling X-ray generation device traveling along a ceiling rail installed on a ceiling of the imaging chamber 300 for example. The X-ray detector 230 detecting a transmission X-ray of an object is provided opposite to the X-ray generation device 220. The operation console 210 has the control device 240 controlling various operations of the X-ray image diagnostic apparatus 200, the operation unit 250, the display unit 260, and the GPS receiver 270.

The control device 240 is comprised of the central control unit 241 controlling various operations as shown in FIG. 13, the region identification unit 242 identifying a country and a region where the X-ray image diagnostic apparatus 200 is installed based on a GPS signal, the display control unit 243 selecting a language used for a GUI and displaying character information on the GUI using the language, the regional information storage unit 244 storing a language used in each country and region as well as regional information that shows standard physique of people who reside in the region, the imaging condition setting unit 245 calculating and setting X-ray conditions based on system information included in the above regional information, the x-ray control unit 246 controlling the X-ray generation device 220, the detector control unit 247 performing an operation to read a transmission X-ray signal generated by the X-ray detector 230, the image processing unit 248 generating an X-ray image of an object based on the transmission X-ray signal, and the image storage unit 249 storing X-ray images.

Next, the processing flow of an X-ray image diagnostic apparatus related to the fourth embodiment will be described based on FIG. 14.

(Step S301)

The main power of the X-ray image diagnostic apparatus 200 is turned on (S301).

(Step S302)

The GPS receiver 270 receives a GPS signal (S302).

(Step S303)

The region identification unit 242 calculates a latitude and a longitude based on a GPS signal to identify a country or a region in which a current position of the mobile X-ray apparatus 1 is included (S303).

(Step S304)

The region identification unit 242 refers to regional data of the regional information storage unit 244, reads out regional information of a country or a region identified in Step S303, and then brings the regional information to the display control unit 243 and the imaging condition setting unit 245 (Step S304). As shown in FIG. 15, the regional information is configured as a table including language (not limited to official languages) information by country and region, information of standard physique such as body thickness, and initial values of imaging conditions according to the physique information.

(Step S305)

The display control unit 243 refers to regional information and displays it on a GUI using a language of each country and region. Also, the imaging condition setting unit 245 refers to regional information, calculates values of X-ray conditions (for example, kV and mA) etc., and then sets the values as the X-ray conditions (S305).

For example, when “Japan” is identified as a region, regional data for Japan is read out from the regional data of FIG. 15, and then Japanese is chosen as a language to be used on a GUI.

Also, the imaging condition setting unit 245 refers to regional information about Japan from among the regional information of FIG. 15, reads out imaging conditions such as X-ray conditions (kV, mA, etc.) in a case where “Body Thickness” is set to “Standard”, and then sets it as an initial value. On the other hand, in a case where standard (average) physique of people in a country and a region other than Japan is greater than that of standard (average) Japanese for example, that is, the body thickness is thick, information showing that “Body Thickness” is “Thick” and X-ray conditions suitable for a standard (average) thickness of people in the said country or region are specified in the table configuring regional information of the said country or region. Therefore, the table of the said country or region is read out to set X-ray conditions specified in the table.

The set X-ray conditions are displayed on the GUI shown in FIG. 8 described above, for example. Then, by operating the adjustment buttons 86 of in FIG. 8 to increase or decrease setting values, automatically input X-ray conditions can be adjusted finely. Additionally, although information showing physique and imaging conditions are specified for regional information in the above description, a table including the information showing physique and a conversion table of imaging conditions according to the physique may be separately provided in the regional information. Then, the imaging condition setting unit 245 may read the information showing physique from the regional information and decide imaging conditions by referring to the conversion table.

In this case, the conversion table comprises a part of the regional information as information associated with the regional information. Additionally, the regional information can include imaging conditions suitable for standard physique in the said country and region in a mode where information showing the standard physique is not included. Also, the above imaging conditions may be subdivided according to not only the physique but the imaging procedure and the imaging site.

According to the present embodiment, a language used on a GUI can be changed using a GPS signal, and imaging conditions (X-ray conditions) can also be automatically input according to physique of people residing in a country and region to which a current position belongs, which can reduce workloads to input imaging conditions (X-ray conditions). Therefore, although there are conventionally many operation steps because an operator such as a manufacturing engineer and a service engineer manually inputs settings and a language to be displayed according to the destination country and region when an X-ray image diagnostic apparatus is shipped, the operation steps can be reduced. Also, although it has been burdensome for users to acquire object information and to set X-ray conditions according to the object at a medical site because the users such as a doctor and a nurse manually input them, X-ray conditions can be set automatically according to the region and country, which can reduce users' workload.

### DESCRIPTION OF REFERENCE NUMERALS

1: mobile X-ray apparatus, 100: imaging support system, 110 and 110a: imaging support server, 120: network, 130: RIS server (order information server)

1. An imaging support system comprising:
a medical image imaging apparatus generating a medical image of an object;
a position detection unit detecting a current position of the medical image imaging apparatus;
an object candidate search unit searching object candidates to be examined on the basis of the current position;
a first operation unit receiving a selection operation of the object to be examined from among the object candidates; and
an imaging condition setting unit setting imaging conditions of the object to be examined.

2. The imaging support system according to claim 1, comprising:
a display unit displaying the set imaging conditions; and
a second operation unit receiving a change operation for the displayed imaging conditions.

3. The imaging support system according to claim 2,
wherein the medical image imaging apparatus is a mobile apparatus moving in a facility, the position detection unit calculates a current position of the mobile apparatus in the facility, and
the object candidate search unit searches the object candidates based on a current position in the facility.

4. The imaging support system according to claim 3, further comprising:
a map information storage unit storing map information in which position information of each point in the facility corresponds to the layout of the facility, wherein the position detection unit refers to the map information and calculates a current position in the facility.

5. The imaging support system according to claim 4, further comprising:
a bed information storage unit storing bed information in which identification information of beds disposed at each point corresponds to position information of each point in the facility;
a bed candidate search unit referring to the bed information, searching beds within a predetermined range including a current position in the facility, and then extracting them as bed candidates; and
an object information storage unit storing object information in which identification information of objects who are using the respective beds corresponds to each of identification information of the beds,
wherein the object candidate search unit refers to the object information to extract identification information of the objects corresponding to identification information of the bed candidates extracted by the bed candidate search unit.

6. The imaging support system according to claim 5, further comprising:
a route storage unit storing a route to perform examination while the mobile apparatus is moving in the facility,
wherein the bed candidate search unit extracts the beds on the route as the bed candidates.

7. The imaging support system according to claim 5, further comprising:
a route prediction unit predicting a route to perform examination while the mobile apparatus is moving in the facility using a current position in the facility and the map information,
wherein the bed candidate search unit extracts the beds on the predicted route as the bed candidates.

8. The imaging support system according to claim 1,
wherein the medical image imaging apparatus is connected to an imaging support server via a network as well as includes a position signal transmission unit transmitting a signal showing a current position of the said medical image imaging apparatus to the imaging support server, the first operation unit, and the imaging condition setting unit,
the imaging support server includes the position detection unit and the object candidate search unit, and
the position detection unit detects a current position of the medical image imaging apparatus based on a signal showing the current position transmitted from the position signal transmission unit.

9. The imaging support system according to claim 1, further comprising:
a regional information storage unit storing regional information showing standard physique of people residing in a country or region where medical image imaging apparatus is installed,
wherein the position detection unit includes a region identification unit identifying a country or region where medical image imaging apparatus is installed, and
the imaging condition setting unit refers to the regional information about a country or region identified by the region identification unit and sets imaging conditions based on the standard physique of people residing in the said country or region.

10. The imaging support system according to claim 9, further comprising:
a display unit displaying the set imaging conditions; and
a display control unit selecting a language to be used for the display screen of the display unit and displaying it on the display screen,
wherein the regional information further includes information about a language used in the country or region, and
the display control unit refers to the regional information about a country or region identified by the region identification unit, selects a language to be used on the display screen based on information about a language used in the said country or region, and then displays the language.

11. A medical image imaging apparatus including an imaging unit that images an object, comprising:
a position detection unit detecting a current position of the medical image imaging apparatus;
an object candidate search unit searching object candidates to be examined on the basis of the current position;
an operation unit receiving a selection operation of the object to be examined from among the object candidates; and
an imaging condition setting unit setting imaging conditions of the object to be examined.
